SEQUENCE LISTING (1 sequence)

SEQ ID NO: 1 - protein, 17 aa, Homo sapiens
EGAFHGDAEALQRPVAS

(19) United States
(12) Patent Application Publication
(10) Pub. No.: US 2005/0271630 A1
(43) Pub. Date: Dec. 8, 2005

(54) Method for treating cancer

(75) Inventors: Shigenori Goto, Tokyo (JP); Kohji Egawa, Tokyo (JP); Toru Kaneko, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MEDINET Co., Ltd., Yokohama-shi (JP)

(21) Appl. No.: 11/059,408

(22) Filed: Feb. 17, 2005

Related U.S. Application Data
(60) Provisional application No. 60/544,322, filed on Feb. 17, 2004.

(51) Int. Cl. A61K 48/00

(52) U.S. Cl. 424/93.7

(57) ABSTRACT

A method for treating cancer having fewer side effects than conventional therapies and capable of improving a response ratio by combining a molecular targeting agent and an immuno-cell therapy.

### CROSS-REFERENCE TO A RELATED APPLICATION

This application is entitled to the benefit of Provisional Patent Application No. 60/544,322, filed on Feb. 17, 2004.

### BACKGROUND OF THE INVENTION

1. Field of the Invention

The present invention relates to a method for treating cancer by a combination of a molecular targeting agent and an immuno-cell therapy.

2. Description of the Background

Conventionally, most methods for treating cancer were surgical operations, radiotherapies or treatments using antitumor agents. In recent years, there has been ongoing research for a therapeutic method that is more effective while maintaining QOL (quality of life) of patients.

In particular, as new therapeutic methods for cancer, a large number of immuno-cell therapies have been carried out. For example, there is an activated autologous lymphocyte transfer therapy in which autologous lymphocytes are cultured and activated ex vivo and are transferred into the body.

These immuno-cell therapies are advantageous in that they produce substantially fewer side effects than conventional cancer therapies and they can keep maintaining QOL of patients.

However, even with these immuno-cell therapies, the complete and partial response ratio is about 10% to 20%, including no change over the long term is still as high as about 30%.

Therefore, several creative trials have been made to achieve a higher therapeutic efficacy by combining chemotherapeutics that suppress cancer cells directly with the immuno-cell therapies.

### SUMMARY OF THE INVENTION

However, the current combination of the chemotherapeutics and the immuno-cell therapies have a problem, that is, the strong myelosuppression caused by the chemotherapeutics itself greatly damages one's immune system, so that the immuno-cell therapy that is combined cannot show enough effects. In view of the above, it is an object of the present invention to provide a new method for treating cancer that has fewer side effects and improves therapeutic efficacy.

In order to achieve the above-mentioned object, a method for treating cancer according to the present invention includes a combination of an immuno-cell therapy and administering a molecular targeting agent.

As one aspect, the immuno-cell therapy includes harvesting lymphocytes from a patient, activating and/or proliferating the lymphocytes, and administering an effective dose of the activated and/or proliferated lymphocytes to the patient. The lymphocytes harvested from the patient can be activated and/or proliferated, for example, by at least one substance selected from the group consisting of an anti-CD3 antibody, IL-2, a DC, an antigen-presenting cell, a cancer antigenic peptide and a protein expressed specifically in a cancer cell. Further, as another aspect, the immuno-cell therapy includes harvesting dendritic cells (DCs) from the patient or differentiating and inducing DCs ex vivo, allowing the DCs to present an antigen, and administering an effective dose of the DCs that are activated to the patient. The DCs can be allowed to present the antigen, for example, by a cancer antigenic peptide and/or a protein expressed specifically in a cancer cell. Specific examples of the immuno-cell therapy include a lymphokine-activated killer cell (LAK) therapy, a cytotoxic T lymphocyte (CTL) therapy, a tumor infiltrating lymphocytes (TIL) therapy, a DC vaccination and a combination thereof.

The administration of the molecular targeting agent includes administering an effective dose of the molecular targeting agent to the patient. The molecular targeting agent targets a molecule selected from the group consisting of an oncogene, a tumor suppressor gene, a molecule associated with a proliferation signal, a molecule associated with a DNA synthesis system or a DNA repair system and a molecule associated with infiltration or metastasis. The molecular targeting agent preferably is a molecular targeting agent targeting the molecule associated with the proliferation signal, more preferably is a tyrosine kinase inhibitor and still more preferably is gefitinib or imatinib mesylate.

As another aspect, the method for treating cancer according to the present invention includes a combination of an immuno-cell therapy and an administration of gefitinib.

The immuno-cell therapy can be the above-described immuno-cell therapies, and the administration of the gefitinib includes administering an effective dose of the gefitinib to the patient. The lymphocytes and/or the DCs in the immuno-cell therapy are administered once every about two to about four weeks, for example, and the gefitinib is administered once every day, for example. The dosage of the gefitinib administered at one time is about 200 mg to about 300 mg, for example.

The immuno-cell therapy preferably is a CD3-LAK therapy. The CD3-LAK therapy includes activating and/or proliferating lymphocytes harvested from a patient by an anti-CD3 antibody and IL-2. The number of the lymphocytes administered at one time is, for example, about 0.3×10⁹ to about 1.0×10¹¹ and preferably is about 2.0×10⁹ to about 1.0×10¹⁰.

As yet another aspect, the method for treating cancer according to the present invention includes a combination of an immuno-cell therapy and an administration of imatinib mesylate.

The immuno-cell therapy can be the above-described immuno-cell therapies, and among them, it preferably is a combination of a CD3-LAK therapy and a DC vaccination. The CD3-LAK therapy includes activating and/or proliferating lymphocytes harvested from a patient by an anti-CD3 antibody and IL-2, and the DC vaccination includes activating immature DCs harvested from the patient by a cancer antigen so as to obtain mature DCs. The activated lymphocytes are administered, for example, once every about two to about four weeks, and the mature DCs are administered, for example, once every about two to about four weeks. The number of the lymphocytes administered at one time is, for example, about 0.3×10⁹ to about 1.0×10¹¹ and preferably is about 2.0×10⁹ to about 1.0×10¹⁰. Further, the number of the DCs administered at one time is, for example, at least about 1.0×10⁷.

The lymphocytes and the DCs may be administered on the same day or different days. In the case of administering on the same day, the lymphocytes and the DCs may be mixed and then administered, or they may be administered in different manners. In the case of administering on different days, the lymphocytes and the DCs may be administered in any order, or the DCs and the lymphocytes may be administered alternately in this order.

The administration of the imatinib mesylate includes administering an effective dose of the imatinib mesylate to the patient. The imatinib mesylate is administered once every day, for example, and the dosage of the imatinib mesylate administered at one time is about 50 mg to about 350 mg, for example.

Currently, so-called “molecular targeting agents,” which inhibit specific molecular receptors that frequently exist in cancer cells for cancer proliferation or the like, have been developed and come into use as a new therapeutic method.

On the other hand, immuno-cell therapies that have become widespread in recent years also have an effect that cannot be obtained by conventional therapeutic methods, for example, produce fewer side effects.

Accordingly, the inventors of the present invention combined these therapeutic methods and found that it was possible to achieve a higher efficacy than the conventional therapeutic methods without canceling out the efficacies of one another, thus completing the method for treating cancer according to the present invention.

As described above, by combining the immuno-cell therapy and the molecular targeting agent having a high therapeutic efficacy, the method for treating cancer according to the present invention can enhance the efficacy of treating cancer, for example, produce fewer side effects than the conventional methods, while maintaining the efficacy of the immuno-cell therapy.

Furthermore, in the case of using the molecular targeting agent alone, the dosage thereof sometimes has to be reduced because of the side effects such as symptoms to skin and digestive organs. Then, such a disadvantage in efficacy can be compensated for by using the immuno-cell therapy as well.

Moreover, since the immuno-cell therapy is usable for all kinds of cancer, the adoption of the suitable molecular targeting agent for each cancer can make the combination treatment actual one without any restriction by the kinds of cancer, the condition of diseases or the like.

### BRIEF DESCRIPTION OF THE DRAWINGS

FIG. 1 is a circle graph showing a treatment result of a treatment method according to the present invention.

FIG. 2 is a CT image showing the condition before conducting the treatment method according to the present invention.

FIG. 3 is a CT image showing the result after conducting the treatment method according to the present invention.

FIG. 4 is a circle graph showing treatment results of the treatment method according to the present invention and a conventional treatment method.

### DESCRIPTION OF THE INVENTION

The following is a description of an embodiment of the present invention.

A method for treating cancer according to the present invention (in the following, referred to as “the method of the present invention”) is characterized by combining an immuno-cell therapy and a molecular targeting agent.

The combination therapy of the immuno-cell therapy and the molecular targeting agent according to the present invention includes at least one administration of the molecular targeting agent from the time four weeks prior to an immunocyte administration to the time four weeks after that in the immuno-cell therapy.

Here, the “immuno-cell therapy” includes various therapies that are known currently and, in particular, means a LAK therapy, a CTL therapy (Ikuko Haruta, et al. Journal of Immunotherapy 19(3) 218-223 (1996)), a TIL therapy (W. Marston Linehan et al. Journal of Clinical Oncology vol. 6, No. 5 (May) 839-853 (1988)), a DC (dendritic cell) vaccination (Riccobon A, et al. American Society of Clinical Oncology. 19 469 (2000)) (G. Murphy, et al. The Prostate 29 371-380 (1996)), etc. These therapies further can be divided according to antibodies and reagents to be used. For example, the LAK therapy includes a LAK therapy in a strict sense, a CD3-LAK therapy, etc.

Further, the “molecular targeting agent” generally refers to agents binding to a specific molecular receptor that exists frequently in a cancer cell, thereby producing an effect of injuring that cancer cell or suppressing the proliferation thereof, and includes agents produced by an antigen-antibody reaction and substances having a function of acting on proteins or the like existing in the cancer cell.

The molecules to be targeted can be an oncogene, a tumor suppressor gene, a molecule contained in a proliferation signal system such as a signal transmission system, a molecular target group contained in a DNA synthesis system or a DNA repair system, an essential molecule for infiltration or metastasis, a molecule concerning vascularization, or the like.

Examples of the molecular targeting agent include gefitinib (trade name: IRESSA™), imatinib mesylate (trade name: GLIVEC™), trastuzumab (trade name: HERCEPTIN™), rituximab (trade name: RITUXAN™), ibritumomab (trade name: ZEVALIN™), tositumomab (trade name: BEXXAR™), bortezomib (trade name: VELCADE PS341™), Lym-1 (trade name: ONCOLYM™), a MMP inhibitor, a VEGF inhibitor, an anti-VEGF inhibitor, angiostatin, endostatin, thalidomide, a farnesyltransferase inhibitor, a cyclin-dependent kinase inhibitor, a protein kinase C (PKC) inhibitor and the like.

However, since the molecular targeting agent is used in combination with the immuno-cell therapy in the method of the present invention, it is preferable that it does not inhibit or decrease a patient's immune function.

When it is impossible to continue administering the molecular targeting agent, for example, when gefitinib has caused an interstitial pneumonia, this combined therapeutic method also will be discontinued.

Here, the “cancer” is not particularly limited but can be any cancers. For example, the “cancer” may include lung cancer, hepatoma, myeloma, lymphoma, pancreatic cancer, prostate cancer, stomach cancer, breast cancer, colon cancer or the like.

The combination of the immuno-cell therapy and the molecular targeting agent used in the method of the present invention is not particularly limited but can be selected suitably according to the condition of each patient.

For example, the CD3-LAK therapy and gefitinib, which is a tyrosine kinase inhibitor, can be used in combination to treat the patient with non-small cell lung carcinoma.

Further, the CD3-LAK therapy, the DC vaccination and imatinib mesylate, which also is a tyrosine kinase inhibitor, can be used in combination to treat the patient with acute lymphoblastic leukemia or gastrointestinal stromal tumor.

Here, lymphocytes for the CD3-LAK therapy and DCs for the DC vaccination may be administered on the same day or different days.

In the case of administering the lymphocytes for the CD3-LAK therapy and the DCs for the DC vaccination on the same day, they may be mixed and then administered. Alternatively, they may be administered separately.

In the case of administering them on different days, they may be administered in any order.

Alternatively, the lymphocytes for the CD3-LAK therapy may be administered after administering the DCs for the DC vaccination. When administering the DCs for the DC vaccination in this way, lymphocytes in the body are induced to CTLs (cytotoxic T lymphocytes). By the CD3-LAK therapy of harvesting and utilizing blood in the body containing these induced CTLs, it is possible to proliferate and activate cancer-specific CTLs, allowing a more effective treatment.

Further, depending on molecules to be targeted, it is possible to address various cancers. For example, among the equivalent tyrosine kinase inhibitors, agents targeting molecules different from those by the above-described agents, such as rituximab or the like, can be used.

How to administer the molecular targeting agent into each patient can be selected suitably from administrations by a drip infusion, an injection and an oral administration according to its form.

The suitable kind of the immuno-cell therapy can be selected from the above-described methods according to the symptom.

It is appropriate that the dosage of immunocytes in one treatment is at least about 0.3×10⁹ lymphocytes, and it is particularly appropriate that the dosage thereof in one treatment ranges from 2.0×10⁹ to 1.0×10¹⁰ lymphocytes. Also, in the case of using DCs, the number of DCs preferably is at least about 1.0×10⁷, though it varies depending on the amount of blood collected from the patient and the method for collecting the same.

Furthermore, the period for carrying out the method of the present invention can be set suitably during the immuno-cell therapy according to the patient's condition.

In a general immuno-cell therapy, autologous immunocytes that have been proliferated and activated ex vivo are usually administered once every two weeks for three months as one course. After that course the frequency of the treatments may be changed into the convenient one for the patients, for example, four-week intervals or several successive courses. Also, during this period, the molecular targeting agent can be administered by setting the dosage, the number of administrations, etc. as necessary.

In the following, the present invention will be described in detail by way of examples. However, the present invention is not limited to these examples.

### EXAMPLE 1

At Seta Clinic and Shin-yokohama Medical Clinic, a treatment was carries out by combining gefitinib and a CD3-LAK therapy of immuno-cell therapies.

The study period was one course consisting of three months, which was a standard treatment period of immuno-cell therapies. During this period, the administration of immunocytes according to the immuno-cell therapy was conducted once every two weeks. One tablet (250 mg) of gefitinib per day was administered orally at one time.

Out of patients who underwent an activated autologous lymphocyte therapy in parallel with an administration of gefitinib from September 2002 to January 2003, 18 cases in which the efficacy of the combination therapy with respect to an evaluable lesion was obtained by imaging were studied retrospectively for therapeutic efficacy. Tables 1 and 2 below show a breakdown of these 18 cases.

TABLE 1
Breakdown of 18 cases of gefitinib combination therapy

| | 18 cases of gefitinib |
|---|---|
| Gender | combination therapy |
| Male/Female | 3/15 |
| Age | |
| 41-50 | 1 |
| 51-60 | 6 |
| 61-70 | 6 |
| 71< | 5 |
| Mean (SD) | 64.6 (9.1) |
| PS | |
| 0 | 6 |
| 1 | 9 |
| 2 | 2 |
| 3 | 0 |
| 4 | 1 |
| Mean (SD) | 0.9 (1.0) |

TABLE 2
Combination therapy of activated autologous lymphocyte therapy
and gefitinib with respect to non-small cell lung carcinoma
at Seta Clinic and Shin-yokohama Medical Clinic

| | | | | | Trace | |
|---|---|---|---|---|---|---|
| | Therapeutic | | | | period | Outcome |
| Case | efficacy | Age | Gender | PS | [day] | at the end |
| 1 | PR | 66 | F | 1 | 60 | Alive |
| 2 | NC | 58 | F | 1 | 34 | Alive |
| 3 | PR | 60 | F | 0 | 49 | Alive |
| 4 | NC | 55 | F | 1 | 63 | Alive |
| 5 | NC | 47 | F | 1 | 126 | Alive |
| 6 | PR | 56 | F | 1 | 72 | Alive |
| 7 | NC | 70 | F | 1 | 74 | Alive |
| 8 | PR | 67 | F | 0 | 70 | Alive |
| 9 | PR | 75 | M | 4 | 34 | Alive |
| 10 | PD | 55 | F | 2 | 43 | Dead |
| 11 | NC | 76 | M | 0 | 28 | Alive |
| 12 | PR | 76 | F | 0 | 42 | Alive |
| 13 | PR | 56 | F | 1 | 27 | Alive |
| 14 | NC | 81 | M | 1 | 88 | Alive |
| 15 | PR | 68 | F | 1 | 89 | Alive |
| 16 | PR | 61 | F | 0 | 93 | Alive |
| 17 | PR | 69 | F | 2 | 62 | Alive |
| 18 | PR | 59 | F | 0 | 21 | Alive |
| Avg. | | 64.6 | | 0.9 | 59.7 | |

Cases 1-8: cases at Seta Clinic

Patients of all the 18 cases suffered from pulmonary adenocarcinoma. As shown in Tables 1 and 2 above, there were more female patients, and this could serve as a factor that increased the therapeutic efficacy of gefitinib.

The age ranged from 48 to 81 and was 64.6 on average. Performance Status (PS) was relatively good: 0 or 1 for 15 cases, 2 for 2 cases and 4 for 1 case. 10 cases had a recurrence after the first operation, and 9 cases, which accounted for a half of all the cases, had no response to chemotherapy before this combination therapy. Out of the 18 cases, 16 cases had distant metastases whose metastatic foci were intrapulmonary metastasis to the other lobe, lymph node, bone, brain, liver, etc., and all the cases had a lung lesion.

In this combination therapy, side effects of skin symptoms (eruption and dry skin) for 16 cases (89%), diarrhea for 6 cases (33%) and nausea for 3 cases (17%) were observed. They were reported to be side effects of gefitinib, and no new side effect caused by the combined use of the activated autologous lymphocytes was observed.

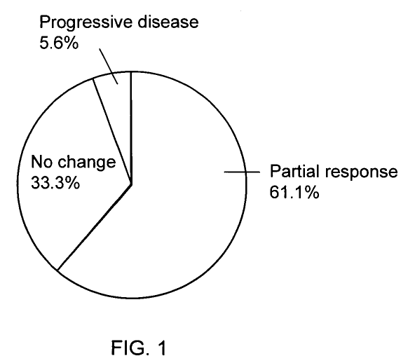

With respect to the therapeutic efficacy, no case showed complete response, 11 cases showed partial response, 6 cases showed no change, and 1 case showed progressive disease. The response ratio (complete response+partial response/the number of evaluated cases) was 61.1%. These results are shown in Table 3 below and a graph in FIG. 1.

TABLE 3
18 cases of combination therapy of activated autologous lymphocyte
therapy and gefitinib with respect to non-small cell lung
carcinoma (from September 2002 to January 2003)

| Treatment | The number | |
|---|---|---|
| result | of cases | Evaluation |
| Complete | 0 | Effective 11 cases |
| response | | (61.1%) |
| Partial | 11 | |
| response | | |
| No change | 6 | No effect 7 cases |
| Progressive | 1 | (38.9%) |
| disease | | |

### EXAMPLE 2

The following is a specific treatment result.

The patient was a 55-year-old woman who was diagnosed in May 2002 as having lung carcinoma (adenocarcinoma), lymphangitic carcinomatosis, carcinomatous pericarditis, multiple intrapulmonary metastases and carcinomatous pleuritis. The treatment history follows.

The first medical examination was carried out on Sep. 19, 2002. From October 4, an activated autologous lymphocyte therapy (CD3-LAK) was started, with the lymphocytes being administered once every two weeks. From October 5, the administration of IRESSA™ (gefitinib) was started. One tablet (250 mg) of gefitinib per day was administered orally at one time. The CD3-LAK therapy was conducted six times at two-week intervals and then continued thereafter at four-week intervals. She showed considerable response by the examination on Feb. 13, 2003 and showed remarkable response by the examination on May 9.

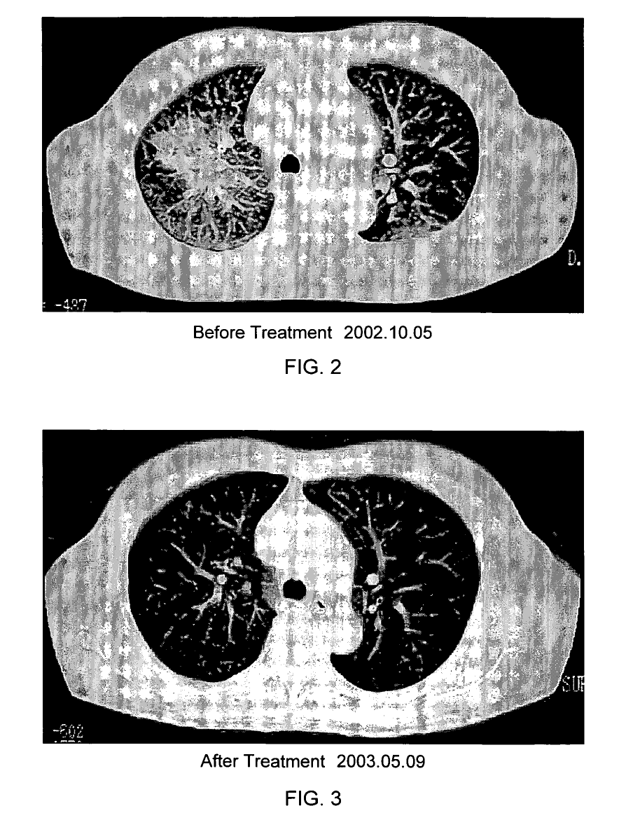

FIGS. 2 and 3 show CT images of the present case. FIG. 2 is a CT image when the combination therapy of CD3-LAK and IRESSA™ administration was started, and FIG. 3 is a CT image at the examination on May 9. As shown in these figures, it was found that the lesioned site that was clearly observed initially almost disappeared.

### EXAMPLE 3

<Comparison with the Case in Combination with the other Chemotherapy>

Next, studies were conducted for comparing a combination therapy with a conventional chemotherapy other than gefitinib and the method of the present invention. Tables 4 and 5 below show 15 cases of the combination group.

TABLE 4
Activated autologous lymphocyte therapy with respect to non-small
cell lung carcinoma (combination therapy) at Seta Clinic

| | | | | | | Trace period | |
|---|---|---|---|---|---|---|---|
| | | | | | | (Trace period of | Outcome |
| | Therapeutic | | | | The number of | combination with | at the |
| Case | efficacy | Age | Gender | PS | administrations | gefitinib) [day] | end |
| 1 | NC(*) | 69 | F | 0 | 6 | 89 | Alive |
| 2 | CR | 41 | M | 2 | 35 | 1195 | Alive |
| 3 | NC | 48 | M | 0 | 6 | 89 | Alive |
| 4 | NC | 20 | M | 1 | 12 | 187 | Alive |
| 5 | PD | 66 | F | 3 | 8 | 143 | Alive |
| 6 | NC | 50 | F | 0 | 16 | 290 | Alive |
| 7 | PR | 44 | M | 0 | 9 | 194 | Alive |
| 8 | PR | 66 | F | 0 | 18 | 743 (60) | Alive |
| 9 | NC | 58 | F | 1 | 7 | 159 (34) | Alive |
| 10 | PR | 60 | F | 0 | 6 | 91(49) | Alive |
| 11 | NC | 55 | F | 1 | 6 | 90(63) | Alive |
| 12 | PR | 47 | F | 0 | 6 | 126(126) | Alive |
| 13 | PR | 56 | F | 1 | 7 | 72(72) | Alive |
| 14 | NC | 70 | F | 1 | 8 | 74(74) | Alive |
| 15 | PR | 67 | F | 0 | 6 | 70(70) | Alive |
| Avg. | | 54.5 | | 0.7 | 10.4 | 240.8 | |

(*)Long no change
Cases 8-15: cases of combining gefitinib

TABLE 5
Breakdown of 15 cases of combination therapy

| | Combination therapy |
|---|---|
| Gender | 15 cases |
| Male/Female | 4/11 |
| Age | |
| -40 | 1 |
| 41-50 | 6 |
| 51-60 | 3 |
| 61-70 | 5 |
| 71-80 | 0 |
| Mean (SD) | 54.5 (13.4) |
| PS | |
| 0 | 8 |
| 1 | 5 |
| 2 | 1 |
| 3 | 1 |
| 4 | 0 |
| Mean (SD) | 0.7 (0.9) |

In Table 4 above, cases 1 to 7 indicate the combination with the conventional chemotherapy, and cases 8 to 15 indicate cases of combining gefitinib corresponding to the treatment method according to the present invention. As shown in Table 5 above, with respect to Performance Status (PS), 8 cases showed 0, 5 cases showed 1, 1 case showed 2 and 1 case showed 3. With respect to the therapeutic efficacy, 1 case showed complete response, 6 cases showed partial response, 7 cases showed no change, and 1 case showed progressive disease. The response ratio was 46.7%. Out of these 7 cases of no change, 1 case showed no change over the long term. The effective ratio including this was 53.3%. The highest number of administrations was 35, and 10.4 times of treatments on average were conducted.

In view of the trace period and the outcome at that time, both of a conventional chemotherapy group and the gefitinib group were shown in Table 4 above.

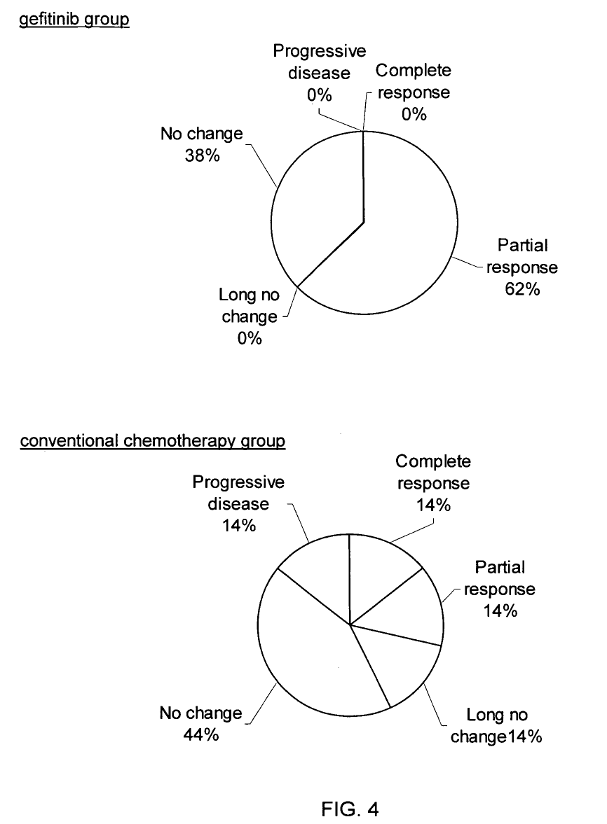

In the gefitinib group, the longest period of survival of 743 days was confirmed, and 7 cases were still alive. In the chemotherapy group, 1 case of complete response corresponds to the patient who has been alive and received treatment without recrudescence for 1195 days up to now. These results are shown in Table 6 below and a graph in FIG. 4.

TABLE 6
15 cases of combination therapy of activated autologous lymphocyte
therapy and gefitinib or activated autologous lymphocyte therapy
and conventional chemotherapy with respect to non-small cell
lung carcinoma (from April 1999 to January 2003)

| | conventional chemotherapy | | gefitinib | |
|---|---|---|---|---|
| Treatment | The number | | The number | |
| result | of cases | Evaluation | of cases | Evaluation |
| Complete | 1 | Effective 3 | 0 | Effective 5 |
| response | | cases | | cases |
| Partial | 1 | (42.8%) | 5 | (62.5%) |
| response | | | | |
| Long no | 1 | | 0 | |
| change | | | | |
| No change | 3 | No effect 4 | 3 | No effect |
| Progressive | 1 | cases | 0 | 3 cases |
| disease | | (57.1%) | | (37.5%) |

As described above, the combination therapy of gefitinib and the immuno-cell therapy showed a higher response ratio than the combination therapy of the other chemotherapy and the immuno-cell therapy. In addition, in the report of 104 cases of Phase 2 study using gefitinib alone, the response ratio was 18%. It was shown that the combination with the immuno-cell therapy provided a more effective cancer therapy.

### EXAMPLE 4

A combination therapy of imatinib mesylate (trade name: GLIVEC™ (registered trademark)) and an immuno-cell therapy was given to three patients below, and the progress was observed.

In the immuno-cell therapy in the treatment below, a CD3-LAK therapy and a DC vaccination were used. DCs were generated as follows.

<Generation of Activated Lymphocytes and DCs>

First, lymphocytes were activated by a usual method. Using VACUTAINER™ (Becton Dickinson, N.J., USA), peripheral blood mononuclear cells were separated from about 22.5 ml of peripheral blood and cultured for two weeks with 700 IU/ml interleukin-2 (IL-2; PROLEUKIN™ (registered trademark), Chiron, Amsterdam) while being stimulated by immobilized anti-CD3 antibody.

After culturing for 14 days, 3×10¹¹ to 10×10¹¹ cells were collected and suspended in 100 ml of saline for injection.

For generating DCs, after mononuclear cells were separated from 45 ml of peripheral blood, floating cells and adherent cells were separated. The obtained adherent cells were cultured for six days with 50 ng/ml of GM-CSF (Primmune Corp., Osaka, Japan) and 50 ng/ml of IL-4, thus obtaining immature DCs. Thereafter, these immature DCs were cultured with antigens for respective tumors to be treated 24 hours prior to administration, thereby obtaining 1×10⁶ to 5×10⁶ mature DCs. Then, these DCs were used for treatment.

Patient 1: 77-Year-Old Man

This patient was diagnosed as having acute lymphoblastic leukemia (ALL) classified as FAB (French-American-British) L3. The treatment history follows.

Since the above diagnosis, he received five courses of AdVP chemotherapy for two months, and showed complete response after the first course and maintained the stable condition in the following four courses.

From the time the above chemotherapy was started, imatinib mesylate was administered. One tablet (200 mg) of imatinib mesylate per day was administered orally at one time.

Six months after the above diagnosis, the combination therapy of the CD3-LAK therapy and the DC vaccination once every two weeks was started. The DCs used here were DCs that were stimulated by p190 minor bcr-abl fusion 17-mer peptide (EGAFHGDAEALQRPVAS: SEQ.ID NO.1).

From the time one year passed since the start of the combination therapy, the dosage of imatinib mesylate was reduced to 100 mg per day, which was administered orally at one time. The side effects were edema of the face, legs and larynx, diarrhea, numbness of the fingers and lassitude.

As a result of the continued combination therapy of the imatinib mesylate, the CD3-LAK therapy and the DC vaccination for 20 months or more, this patient showed complete response.

Patient 2: 66-Year-Old Woman

At the first medical examination, this patient had a large abdominal tumor involving the diaphragm and was diagnosed as having GIST (gastrointestinal stromal tumors). She underwent a gastrectomy.

Two years after the first medical examination, she had a recurrence of mediastinal tumor. Four months later, at the request of the patient, an herbal remedy and some other substitute therapies were started. However, during this time, the tumor progressed markedly.

From the time two years and nine months passed since the first medical examination, the combination therapy of the imatinib mesylate, the CD3-LAK therapy and the DC vaccination was started. At that time, she had a tumor with a diameter of 21 cm in the left hypochondriac region. One tablet (200 mg) of imatinib mesylate per day was administered orally at one time. The frequency of the immuno-cell therapy was once every two weeks initially and once every two to eight weeks from the time one year and four months passed since the initiation of combination therapy. A total of 16 treatments was conducted.

With the combination therapy, the tumor size in the CT image varied as follows.

Before starting combination therapy: 177.56 mm×117.65 mm

Three months after starting combination therapy: 117.65 mm×79.41 mm

Five months after starting combination therapy: 70.11 mm×55.32 mm

One year after starting combination therapy: 65.34 mm×44.23 mm

The CA12-5 level was 151 U/ml at the time of starting the combination therapy and decreased to 26 U/ml three months after starting the therapy.

Although, as the side effects, she experienced itching with eczema of the face and limbs and felt stomach discomfort immediately after the imatinib mesylate administration, she showed partial response lasting for as long as 21 months since the start of the combination therapy. Incidentally, as other side effects, diarrhea and increase in liver enzyme levels (58 U/ml GOT and 43 U/ml GPT) were observed.

Patient 3: 47-Year-Old Man

This patient had a submucosal tumor in the cardiac part of the stomach and underwent a gastrectomy. He was diagnosed as having GIST (gastrointestinal stromal tumors). He underwent four tumor excision surgeries and microwave solidification of liver metastases one year and three months, one year and ten months, three years and one month and three years and ten months after the gastrectomy. 100 to 300 mg (suitably changed depending on the condition of the patient) of imatinib mesylate per day was administered orally at one time. At the time of administration, he showed side effects on his digestive organs.

Three years and three months after the gastrectomy, the combination therapy of the CD3-LAK therapy, the DC vaccination and the imatinib mesylate was started. One tablet (200 mg) of imatinib mesylate per day was administered orally at one time.

The progress is as follows. MRI taken one year and one month after starting the combination therapy showed two foci, one of 1.1 cm and the other of 0.6 cm in size. One month later, the foci decreased slightly. The interpretation of PET performed one year and five months after starting the therapy showed no extraordinary accumulation.

Although diarrhea was observed as the side effects, the tumor had not progressed for 25 months since the combination therapy was started.

From the results described above, it was shown that the combination therapy of the immuno-cell therapy and the imatinib mesylate also was highly effective as the treatment method according to the present application. Further, although some side effects were observed in the above-described cases, they were caused by imatinib mesylate and not by the immuno-cell therapy.

As described above, the method for treating cancer according to the present invention combines an immuno-cell therapy and a molecular targeting agent, thereby providing a treatment that is more effective than a treatment of individually performing one of the above.

The invention may be embodied in other specific forms without departing from the spirit or essential characteristics thereof The embodiments disclosed in this application are to be considered in all respects as illustrative and not restrictive, the scope of the invention being indicated by the appended claims rather than by the foregoing description, all changes that come within the meaning and range of equivalency of the claims are intended to be embraced therein.

1. A method for treating cancer in a patient comprising:
providing an immuno-cell therapy and administering a molecular targeting agent to the patient in an amount sufficient to treat the patient.

2. The method for treating cancer according to claim 1, wherein the immuno-cell therapy comprises
harvesting lymphocytes from the patient,
activating and/or proliferating the lymphocytes, and
administering an effective dose of the activated and/or proliferated lymphocytes to the patient.

3. The method for treating cancer according to claim 1, wherein the immuno-cell therapy comprises
harvesting dendritic cells (DCs) from the patient or differentiating and inducing DCs ex vivo,
allowing the DCs to present an antigen, and
administering an effective dose of the DCs presenting the antigen to the patient.

4. The method for treating cancer according to claim 1, wherein the immuno-cell therapy comprises
harvesting lymphocytes from the patient,
activating and/or proliferating the lymphocytes,
administering an effective dose of the activated and/or proliferated lymphocytes to the patient,
harvesting dendritic cells (DCs) from the patient or differentiating and inducing DCs ex vivo,
allowing the DCs to present an antigen, and
administering an effective dose of the DCs presenting the antigen to the patient.

5. The method for treating cancer according to claim 1, wherein the immuno-cell therapy comprises at least one therapy selected from the group consisting of a lymphokine-activated killer cell (LAK) therapy, a cytotoxic T lymphocyte (CTL) therapy, a tumor infiltrating lymphocytes (TIL) therapy and a dendritic cell (DC) vaccination.

6. The method for treating cancer according to claim 2, wherein the lymphocytes harvested from the patient are activated and/or proliferated by at least one substance selected from the group consisting of an anti-CD3 antibody, IL-2, a DC, an antigen-presenting cell, a cancer antigenic peptide and a protein expressed specifically in a cancer cell.

7. The method for treating cancer according to claim 3, wherein the antigen presented by the DCs is a cancer antigenic peptide and/or a protein expressed specifically in a cancer cell.

8. The method for treating cancer according to claim 1, wherein the molecular targeting agent targets a molecule selected from the group consisting of an oncogene, a tumor suppressor gene, a molecule associated with a proliferation signal, a molecule associated with a DNA synthesis system, a molecule associated with a DNA repair system and a molecule associated with infiltration or metastasis.

9. The method for treating cancer according to claim 8, wherein the molecular targeting agent targets the molecule associated with the proliferation signal.

10. The method for treating cancer according to claim 8, wherein the molecular targeting agent is a tyrosine kinase inhibitor.

11. The method for treating cancer according to claim 10, wherein the tyrosine kinase inhibitor is gefitinib or imatinib mesylate.

12. A method for treating cancer in a patient comprising:
providing an immuno-cell therapy and administering gefitinib to the patient in an amount sufficient to treat the patient.

13. The method for treating cancer according to claim 12, wherein the immuno-cell therapy comprises
harvesting lymphocytes from the patient,
activating and/or proliferating the lymphocytes, and
administering an effective dose of the activated and/or proliferated lymphocytes to the patient.

14. The method for treating cancer according to claim 12, wherein the immuno-cell therapy comprises
harvesting dendritic cells (DCs) from the patient or differentiating and inducing DCs ex vivo,
allowing the DCs to present an antigen, and
administering an effective dose of the DCs presenting the antigen to the patient.

15. The method for treating cancer according to claim 12, wherein the immuno-cell therapy comprises
harvesting lymphocytes from the patient,
activating and/or proliferating the lymphocytes,
administering an effective dose of the activated and/or proliferated lymphocytes to the patient,
harvesting dendritic cells (DCs) from the patient or differentiating and inducing DCs ex vivo,
allowing the DCs to present an antigen, and
administering an effective dose of the DCs presenting the antigen to the patient.

16. The method for treating cancer according to claim 12, wherein lymphocytes and/or DCs of the immuno-cell therapy are administered once every about two to about four weeks, and the gefitinib is administered once every day.

17. The method for treating cancer according to claim 12, wherein the immuno-cell therapy comprises a CD3-LAK therapy.

18. The method for treating cancer according to claim 17, wherein the lymphocytes harvested from a patient are activated and/or proliferated by contacting the lymphocytes with an anti-CD3 antibody and IL-2.

19. The method for treating cancer according to claim 18, wherein the number of the activated and/or proliferated lymphocytes administered at one time is about 0.3×10⁹ to about 1.0×10¹¹.

20. The method for treating cancer according to claim 18, wherein the number of the activated and/or proliferated lymphocytes administered at one time is about 2.0×10⁹ to about 1.0×10¹⁰.

21. The method for treating cancer according to claim 12, wherein a dosage of the gefitinib administered at one time is about 200 mg to about 300 mg.

22. A method for treating cancer in a patient comprising:
providing an immuno-cell therapy and administering imatinib mesylate to the patient in an amount sufficient to treat the patient.

23. The method for treating cancer according to claim 22, wherein the immuno-cell therapy comprises
harvesting lymphocytes from the patient,
activating and/or proliferating the lymphocytes, and
administering an effective dose of the activated and/or proliferated lymphocytes to the patient.

24. The method for treating cancer according to claim 22, wherein the immuno-cell therapy comprises
harvesting dendritic cells (DCs) from the patient or differentiating and inducing DCs ex vivo,
allowing the DCs to present an antigen, and
administering an effective dose of the DCs presenting the antigen to the patient.

25. The method for treating cancer according to claim 22, wherein the immuno-cell therapy comprises
harvesting lymphocytes from the patient,
activating and/or proliferating the lymphocytes,
administering an effective dose of the activated and/or proliferated lymphocytes to the patient,
harvesting dendritic cells (DCs) from the patient or differentiating and inducing DCs ex vivo,
allowing the DCs to present an antigen, and
administering an effective dose of the DCs presenting the antigen to the patient.

26. The method for treating cancer according to claim 22, wherein the immuno-cell therapy comprises a CD3-LAK therapy and a DC vaccination.

27. The method for treating cancer according to claim 26, wherein the lymphocytes harvested from a patient are activated and/or proliferated by contacting the lymphocytes with an anti-CD3 antibody and IL-2.

28. The method for treating cancer according to claim 26, wherein the DC vaccination comprises activating immature DCs harvested from the patient by contacting the immature DCs with a cancer antigen so as to obtain mature DCs.

29. The method for treating cancer according to claim 27, wherein the activated and/or proliferated lymphocytes are administered once every about two to about four weeks,

30. The method for treating cancer according to claim 28, wherein the mature DCs are administered once every about two to about four weeks

31. The method for treating cancer according to claim 26, wherein the imatinib mesylate is administered once every day.

32. The method for treating cancer according to claim 26, wherein lymphocytes of the CD3-LAK therapy and DCs of the DC vaccination are administered on the same day or different days.

33. The method for treating cancer according to claim 32, wherein the lymphocytes and the DCs are mixed and then administered on the same day.

34. The method for treating cancer according to claim 32, wherein the lymphocytes and the DCs are administered in different manners on the same day.

35. The method for treating cancer according to claim 32, wherein the lymphocytes and the DCs are administered in any order on different days.

36. The method for treating cancer according to claim 32, wherein the DCs and the lymphocytes are administered alternately on different days, wherein the DCs are administered first.

37. The method for treating cancer according to claim 29, wherein the number of the activated and/or proliferated lymphocytes administered at one time is about 0.3×10⁹ to about 1.0×10¹¹.

38. The method for treating cancer according to claim 29, wherein the number of the activated and/or proliferated lymphocytes administered at one time is about 2.0×10⁹ to about 1.0×10¹⁰.

38. The method for treating cancer according to claim 30, wherein the number of the mature DCs administered at one time is at least about 1.0×10⁷.

40. The method for treating cancer according to claim 31, wherein a dosage of the imatinib mesylate administered at one time is about 50 mg to about 350 mg.
